(19) United States
(12) Patent Application Publication
(10) Pub. No.: US 2005/0137510 A1
(43) Pub. Date: Jun. 23, 2005

(54) Removable and adjustable, multi-component connection element, and orthosis containing such an element

(75) Inventor: Gerald Dauny, Savigny Les Beaune (FR)

Correspondence Address: SUGHRUE MION, PLLC, 2100 PENNSYLVANIA AVENUE, N.W., SUITE 800, WASHINGTON, DC, 20037, US

(73) Assignee: ETABLISSEMENTS PROTEOR

(21) Appl. No.: 11/009,060

(22) Filed: Dec. 13, 2004

(30) Foreign Application Priority Data
Dec. 12, 2003 (FR) 0314606

(51) Int. Cl. A61F 5/00

(52) U.S. Cl. 602/23

(57) ABSTRACT

The invention relates to a removable and adjustable, multi-component connection element for connecting two parts of an orthosis, comprising
at least two cylindrical rods (1, 2) made of an elastic rigid material, and
two jaws (3, 4), formed in each case by a base part (3a, 4a) with a means (5) for fastening it to part of an orthosis, and at least two grooves (6, 7) intended to receive an end of each of the rods (1, 2), and by a stirrup (3b, 4b) intended to be fixed, by a tightening means (10), to the base part, in the grooves of which the ends of the rods are accommodated, so as to clamp the rods in a position such that their free central parts, not engaged between the base parts and the stirrup of the jaws, are situated in the same plane and are preferably substantially parallel to one another, and to an orthosis containing such an element.

The invention relates to a removable and adjustable, multi-component connection element, to the use of such a connection element for increasing the elasticity of an orthosis in terms of torsion and flexion, and also to an orthosis, in particular a corset for dynamic three-dimensional reduction of scoliosis, containing at least one such connection element.

In the field of orthotic appliances, a problem generally encountered is that of finding a compromise for supporting part of the human body in a way which is rigid and at the same time flexible (elastic). Splints or corsets, for example, are intended to substantially orient the movement of part of the body about an articulation or to impose on this part of the body a certain constraint with a view to correcting a movement or a pathological posture.

Strict immobilization or strong constraint limiting the patient's freedom of movement are generally considered to be disagreeable. This disagreeable feeling could in some cases be attenuated if suitable elastic parts or materials were used permitting a controlled freedom of movement in certain spatial directions while imposing, in other spatial directions, stronger mechanical constraints indispensable to the therapeutic effect.

In its European patent EP 0 389 379 B1, the Applicant has proposed a corset for dynamic three-dimensional reduction of scoliosis which, during the inhalation phase of the patient's breathing cycle, counters the natural expansion of the patient's thoracic cage to the front, thus forcing it to arch, and this brings the dorsal spine into a position of kyphosis, thereby normalizing the vertebral position.

This corset comprises, on the one hand, two lateral brackets made of an elastically deformable plastic material and intended to laterally engage the patient's thorax, with an anterior part disposed in front of the thorax and a posterior part disposed to the rear of the latter, and, on the other hand, a pelvic belt intended to be secured to the patient's pelvis, and at least two lateral struts, or stays, which are made of an elastically deformable semi-rigid material and which join, in the vertical direction, each of said brackets and the corresponding part of the pelvic belt. These struts, made of an elastic rigid material, for example a thermoplastic resin reinforced with carbon fibres, exert a torsional restoring force on the posterior parts of the brackets when these move away from one another in the inhalation phase of the patient's thorax.

However, these struts do not permit any forward or rearward movement (sagittal flexion) of the corset and thus force on the patient a somewhat unnatural posture, which is poorly tolerated by children in particular. However, this strong sagittal rigidity is not indispensable to the corrective action of the corset, and a certain yielding of the corset in the forward/rearward direction could enhance patient comfort and help patients better tolerate the orthosis, but without impairing the therapeutic effect.

With such an aim of providing elastic yield in such a corset, but also in other orthotic appliances, the Applicant has developed a connection element which, when fitted in a suitable position of an orthosis, is able to confer on the latter a certain degree of elasticity in torsion and flexion in a first spatial direction, while maintaining considerable rigidity in a second spatial direction perpendicular to the first.

Consequently, the subject of the invention is a multi-component connection element for connecting two parts of an orthosis, comprising
at least two cylindrical rods made of an elastic rigid material chosen from metal alloys or resin/carbon fibre composites, and
two jaws, each jaw being formed by
   a base part with a means for fastening it to part of an orthosis, and at least two grooves intended to receive an end of each of the rods, and
   a stirrup intended to be fixed, by a tightening means, to the base part, in the grooves of which the ends of the rods are accommodated, so as to clamp the rods in a position such that their free central parts, not engaged between the base parts and the stirrups of the jaws, are situated in the same plane and are preferably substantially parallel to one another.

To describe in more detail the mechanical properties of the connection element of the present invention, it is necessary to define the three basic elastic movements which the connection element can undergo in response to a stress:
The first of these movements is lateral flexion, namely the flexion of the rods in the plane passing through the central axes of all the rods.
The second movement is sagittal flexion, namely the flexion of the rods in a plane parallel to the rods and perpendicular to the plane of lateral flexion, and finally
The horizontal torsion resulting from a rotational stress of one of the jaws relative to the other in a plane perpendicular to the rods, causing these to twist.

The connection element of the present invention effects an elastic sagittal flexion and an elastic horizontal torsion under relatively moderate stresses, but offers substantial resistance to lateral flexion.

The advantage of this novel connection element lies not only in its anisotropic elastic properties, but also in the fact that it can be easily removed and adjusted by the orthotist, for example by simply moving the jaws away from one another or closer together, or by replacement and/or addition of one or more rods.

The elastic behaviour of the connection element will depend on, among other things, the elastic properties of the material forming the rods (Young's modulus, elastic elongation), the free length of the rods, the diameter of the rods, the number of rods mounted in the same plane, or the distance between the rods.

The elasticity of the material, the free length of the rods and the diameter of the rods have an influence on the three basic movements described above. The system will be all the more flexible in the three spatial directions (lateral flexion, sagittal flexion and horizontal torsion), the more elastic the material, the longer the free length of the rods, and the smaller the diameter of the rods. The orthotist will thus be able to adapt the properties of the element by varying these parameters and by possibly combining different rods. Increasing the number of rods above 2 greatly increases the rigidity in lateral flexion, moderately increases the torsional rigidity, and increases only slightly the rigidity in sagittal flexion. Modifying the distance between the rods has no influence on sagittal flexion and on lateral flexion, but greatly influences horizontal torsion.

Bending the rods before or during tightening of the stirrups on the base parts of the jaws affords other possibilities of adjusting the dimensions and mechanical properties of the connection element of the present invention. The rods can, for example, be bent, in the plane of sagittal flexion, beyond the elastic deformation of the material, or can be bent, in the plane of lateral flexion, within or beyond elastic deformation before tightening of the stirrups. The convex rod then assumes a free length greater than the concave rod. Finally, horizontal torsion can be imposed on the rods before the stirrups are tightened.

The material forming the elastic cylindrical rods must be both sufficiently rigid and elastic to satisfy its function as a flexible join in a system for forced support of part of the human body.

Examples of suitable materials are metal alloys, for example spring steel, or superelastic alloys, or composites of thermoplastic or thermosetting resins and carbon fibres, these latter preferably being braided or woven.

The Applicant has found that the connection element satisfies its function as a flexible join particularly effectively when the elastic material forming the rods has an elastic elongation of between 1 and 15%, preferably between 2 and 8%, which is generally the case with the superelastic alloys mentioned above.

The ends of the cylindrical rods made of an elastic rigid material must be perfectly fixed between the base part and the stirrup of each jaw. The tightening means for fixing the stirrup securely on the base part is preferably a screw in a threaded orifice provided in the stirrup and at a corresponding position in the base part.

As has been indicated above, the ends of the cylindrical rods are accommodated in grooves provided for this purpose in each of the base parts of the jaws. These grooves preferably have a depth less than the diameter of the rods in such a way that only part of the thickness of the rods disappears in said grooves and another part of the thickness protrudes beyond the plane surface of the base part of each jaw. The stirrup will then comprise grooves corresponding to those of the base part. The depth of the grooves in the base part and in the stirrup is preferably slightly less than ½ the diameter of the rods.

The rod ends and the grooves in the base part and in the stirrup can be perfectly straight, which affords the advantage of permitting reversible adjustment of the free length of the rods at any time, by simply loosening and then retightening the tightening means. In this embodiment, however, the risk of the rods being torn out from the jaw is not inconsiderable, because the ends of the rods can slide in the grooves under the effect of a substantial pulling stress.

In a preferred embodiment of the connection element of the present invention, this problem is solved by virtue of the rods having bent ends. The “hook” effect of a bend provided at each of the ends of the rods will keep these firmly secured between the base parts and the stirrups of the jaws, provided of course that the grooves intended to receive these ends of the rods have a corresponding bent shape.

An equivalent “hook” effect is obtained if two rods are joined at their ends via a U-shaped rounded part, and if two grooves, intended to receive these rod ends joined in a U-shape, have a corresponding U-shaped rounded part. This, therefore, constitutes another preferred embodiment of the connection element of the present invention.

In yet another preferred embodiment of the present invention, the connection element has a configuration that can be adapted by virtue of a large number of grooves provided on several faces of each of the jaws. More precisely, in this embodiment the base part of at least one of the jaws has a polyhedron shape, preferably a rectangular parallelepiped, supporting, on one of its faces, a securing means and, on several of its other faces, at least two parallel grooves intended to receive the ends of the rods. Depending on the orientation and offset he wishes to give to the securing means, the orthotist will then be able to choose the face of the base part which is to receive the ends of the rods and against which the stirrup will be applied. In a particularly preferred manner, each of the 2, 3, 4 or 5 other faces of the parallelepipedal base part of the jaw comprises not just a single pair of parallel grooves, but two pairs of parallel grooves forming a right angle with respect to one another.

As has been described above in detail, the particular structure of the connection element of the present invention, formed by at least two elastic rods clamped between two jaws, means that this element has elastic properties which vary as a function of the direction of the stress applied. This property can be exploited in order to selectively adapt the rigidity of an orthosis.

Consequently, the invention also relates to the use of a connection element, such as described above, for adjustably increasing the elasticity of an orthosis in torsion and in flexion in a particular plane while maintaining rigidity in another plane perpendicular to the first one. To do this, one or more connection elements according to the invention are mounted preferably in such a way that the planes of sagittal flexion of the various elements are substantially parallel to one another and, in the case of a corset, parallel to the sagittal plane of the patient.

An example of a preferred application of a connection element according to the invention is its use in a corset for dynamic three-dimensional reduction of scoliosis, as described in patent EP 0 389 379 B1 from the Applicant. In this case, two connection elements according to the invention are mounted respectively as integral parts of each of the rigid lateral struts of the corset and are oriented such that their plane of sagittal flexion is substantially parallel to the sagittal plane of the patient (forward/rearward flexion plane). In other words, each of the two struts of the known corset is replaced by two half-struts which are fixed respectively to the pelvic belt and to a lateral bracket and are connected to one another by the connection element according to the invention. Such an assembly permits elastic flexion in the sagittal plane of the patient while ensuring rigidity in the lateral plane of the patient.

Finally, a third and last subject of the invention is an orthosis comprising at least one connection element such as described above, and in particular a corset for dynamic three-dimensional reduction of scoliosis, of the type described in patent EP 0 389 379 B1 from the Applicant, comprising
on the one hand, two lateral brackets made of an elastically deformable plastic material and intended to laterally engage the patient's thorax, with an anterior part disposed in front of the thorax, and a posterior part disposed to the rear of the latter,
on the other hand, a pelvic belt intended to be secured to the patient's pelvis, and
at least two lateral struts which are made of an elastically deformable semi-rigid material and which join, in the vertical direction, each of said brackets and the corresponding part of the pelvic belt,
   this corset being characterized by the fact that a connection element according to the invention is fitted, as an integral part of each of the lateral struts, in the free part thereof, and is oriented in such a way that the lateral and sagittal planes of said connection element are substantially parallel respectively to the lateral and sagittal planes of the patient, so as to allow the latter a certain freedom in terms of horizontal torsion and sagittal flexion.

Other possible applications of the connection element according to the invention are, for example, head supports and corsets for rehabilitation of posture.

The invention is now described with reference to the attached non-limiting drawings in which:

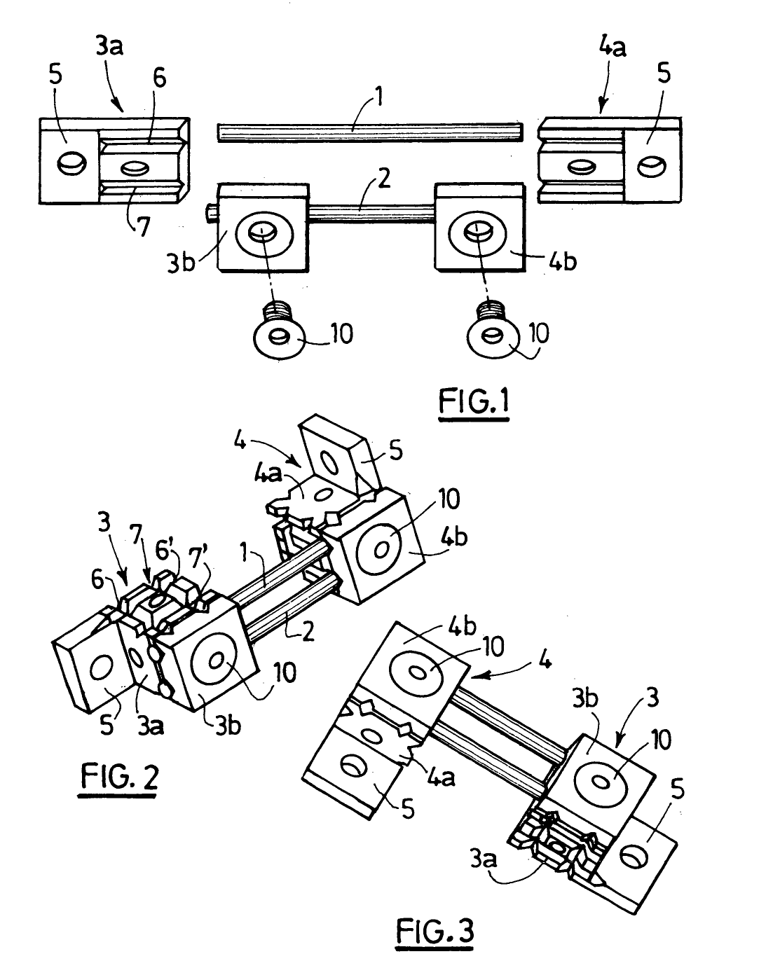

FIG. 1 is an exploded perspective view of a connection element according to the invention,

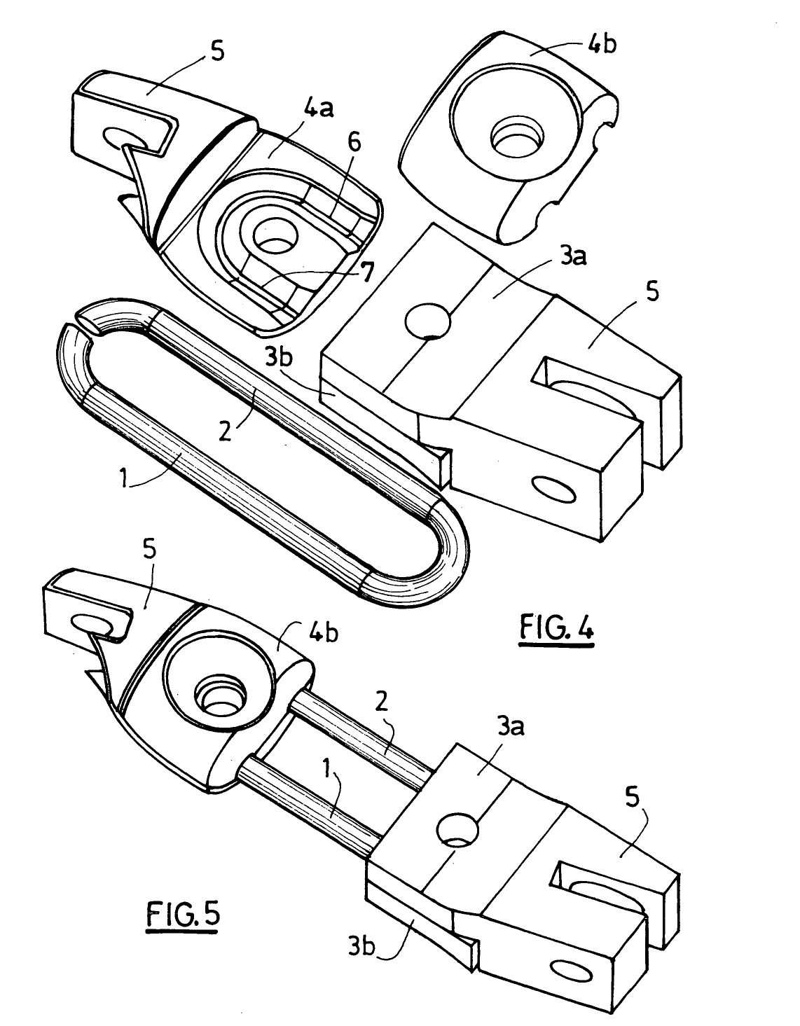

FIGS. 2 and 3 are perspective views of a connection element with adjustable jaws according to a preferred embodiment of the invention,

FIG. 4 is a perspective view of the various parts forming a connection element according to another preferred embodiment of the invention,

FIG. 5 is a perspective view of a connection element assembled from the parts in FIG. 4,

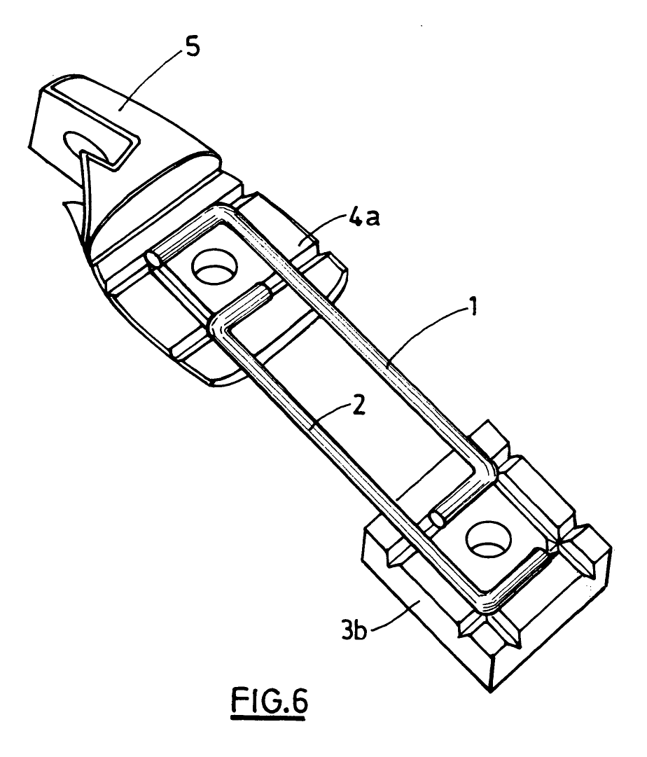

FIG. 6 is a perspective view of a part of a connection element according to a third preferred embodiment according to the invention,

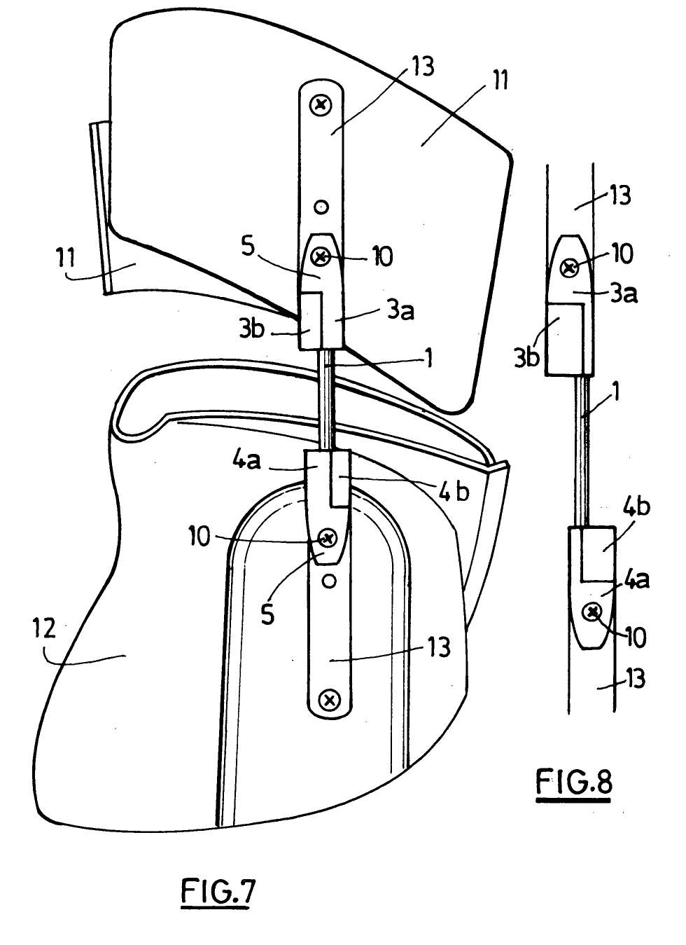

FIG. 7 is a side view of a connection element according to the invention incorporated in a corset as an integral part of one of the lateral struts, and

FIG. 8 is a side view of a variant of a connection element according to the invention as an integral part of one of the lateral struts of a corset.

The various parts which, after being assembled, form the connection element of the present invention are represented in FIG. 1. Each connection element comprises two jaws 3, 4, formed in each case by a base part 3a, 4a and by a stirrup 3b, 4b. Each base part 3a, 4a is composed of a part 5 for fastening to the corset and with a threaded hole, and of a part comprising two straight grooves 6, 7 which are intended to receive the ends of two rods 1, 2. The two stirrups 3b, 4b also comprise, on one of their faces, two straight grooves 6′, 7′ which, after the stirrup has been secured to the base part, will be located in a position corresponding to that of the grooves 6, 7 of the base part, so as to securely clamp the ends of the two rods. The stirrups 3b, 4b are secured on the base parts 3a, 4a by means of a screw 10 screwed into holes 9a, 9b provided in the stirrup and base part, respectively.

The connection element shown in FIGS. 2 and 3 differs from that in FIG. 1 mainly in terms of the geometry of the two base parts 3a, 4a. These are substantially cuboid and support, on one of their surfaces, a part 5 used for fastening to the corset and with a drilled hole. Four of the five other surfaces of the cube each support two pairs of parallel grooves 6, 7, forming a right angle with respect to one another. Given that each pair of grooves 6, 7 can receive one pair of rods 1, 2 in two different ways (the free part of the rods protruding from the jaw at one side or the other), there are in total sixteen ways of fastening a jaw to two fixed rods, with the same number of different orientations of the fastening part 5. In FIGS. 2 and 3, the jaw 3 is fixed identically to the rods 1, 2, while the relative position of the jaw 4 with respect to the jaw 3 is different.

FIGS. 4 and 5 illustrate an embodiment of the connection element of the present invention in which two rods 1, 2 are joined at their ends by a U-shaped rounded part. Such a rounded part, whether open or closed, provides a hook effect which prevents the rods 1, 2 from sliding in the grooves 6, 7 in the event of excessive traction. The two jaws are simpler and lighter than those shown in FIGS. 2 and 3 but impose a unique and fixed orientation on the fastening parts 5.

FIG. 6 illustrates an embodiment of the connection element of the present invention in which the two rods 1, 2 are bent at their ends and are accommodated in corresponding grooves of the jaws. A hook effect analogous to that described for FIGS. 4 and 5 is obtained.

FIG. 7 shows a connection element fitted in the area of the free central part of a lateral strut 13 of a corset for dynamic three-dimensional reduction of scoliosis, joining the pelvic belt 12 to one of the lateral brackets 11. The connection element is made up of two jaws, these being formed in each case by a base part 3a, 4a and by a stirrup 3b, 4b, and two rods 1, 2, only one of which is visible. In this position, the plane of sagittal flexion of the connection element is substantially parallel to the sagittal plane of symmetry of the patient. The presence and position of this connection element in the corset increases the flexibility of the latter in the event of the patient bending forward or backward or in the event of lateral torsion of the thorax, which is engaged between the lateral brackets, relative to the pelvic belt secured on the patient's pelvis. The quite substantial rigidity of the connection element in the plane of lateral flexion, parallel to the plane of lateral flexion of the patient, nevertheless allows the assembly to maintain sufficient rigidity for effective support of the thorax by the two support brackets.

Finally, FIG. 8 shows, analogously to FIG. 7, a connection element according to the invention fitted in the area of the free central part of a lateral strut. The geometry of the jaws, more precisely the small thickness of the base parts 3a, 4a and the large thickness of the stirrups 3b, 4b, results in an offset arrangement of one of the half-struts relative to the other, and thus results in the lateral bracket (not shown), on which the upper half-strut is fixed, being offset to the front or to the rear in relation to the pelvic belt (not shown) on which the lower half-strut is fixed.

1. Removable and adjustable, multi-component connection element for connecting two parts of an orthosis, comprising
at least two cylindrical rods (1, 2) made of an elastic rigid material chosen from metal alloys or resin/carbon fibre composites, and
two jaws (3, 4), each jaw being formed by
a base part (3a, 4a) with a means (5) for fastening it to part of an orthosis, and at least two grooves (6, 7) intended to receive an end of each of the rods (1, 2), and
a stirrup (3b, 4b) intended to be fixed, by a tightening means (10), to the base part (3a, 4a), in the grooves of which the ends of the rods (1, 2) are accommodated, so as to clamp the rods in a position such that their free central parts, not engaged between the base parts (3a, 4a) and the stirrups (3b, 4b) of the jaws, are situated in the same plane and are preferably substantially parallel to one another.

2. Connection element according to claim 1, characterized in that the material forming the rods (1, 2) has an elastic elongation of between 1 and 15%, preferably between 2 and 8%.

3. Connection element according to either of the preceding claims, characterized in that the base part (3a, 4a) of at least one of the jaws has a polyhedron shape supporting, on one of its faces, a fastening means (5) and, on several of its other faces, at least two parallel grooves (6, 7) intended to receive the ends of the rods (1, 2).

4. Connection element according to claim 3, characterized in that the base part (3a, 4a) of at least one of the jaws is a rectangular parallelepiped supporting, on one of its faces, a fastening means (5) and, on each of the 2, 3, 4 or 5 other faces, two pairs of grooves, the two grooves of each pair being parallel to one another, and the two pairs of grooves forming a right angle with respect to one another.

5. Connection element according to any one of the preceding claims, characterized in that the ends of the rods have a bent shape, and in that the grooves intended to receive these ends of the rods have a corresponding bent shape.

6. Connection element according to any one of claims 1 to 4, characterized in that two rods are joined at their ends by way of a U-shaped rounded part, and in that two grooves, intended to receive these rod ends joined in a U shape, have a corresponding U-shaped rounded form.

7. Orthosis comprising at least one connection element according to any one of claims 1 to 6.

8. Orthosis according to claim 7, characterized in that it is a corset for dynamic three-dimensional reduction of scoliosis, comprising
on the one hand, two lateral brackets (11) made of an elastically deformable plastic material and intended to laterally engage the patient's thorax, with an anterior part disposed in front of the thorax, and a posterior part disposed to the rear of the latter,
on the other hand, a pelvic belt (12) intended to be secured on the patient's pelvis, and
at least two lateral struts (13) which are made of an elastically deformable semi-rigid material and which join, in the vertical direction, each of said brackets (11) and the corresponding part of the pelvic belt (12),
a connection element according to any one of claims 1 to 6 being fitted, as an integral part of each of the lateral struts (13), in the free part thereof, and being oriented in such a way that the lateral and sagittal planes of said connection element are substantially parallel respectively to the lateral and sagittal planes of the patient, so as to allow the latter a certain freedom in terms of horizontal torsion and sagittal flexion.
